(19) United States
(12) Patent Application Publication
(10) Pub. No.: US 2018/0055740 A1
(43) Pub. Date: Mar. 1, 2018

(54) ENTERAL FEEDING TUBE

(71) Applicant: Shailinder Jit Singh, Nottingham (GB)

(72) Inventor: Shailinder Jit Singh, Nottingham (GB)

(21) Appl. No.: 15/607,860

(22) Filed: May 30, 2017

(30) Foreign Application Priority Data
Sep. 1, 2016 (GB) 1614865.2

(51) Int. Cl. A61J 15/00; A61N 1/05

(52) U.S. Cl. CPC A61J 15/0084; A61J 15/0015; A61J 15/0011; A61B 5/0084; A61J 15/0069; A61J 15/0019; A61N 1/0509; A61J 15/0073

(57) ABSTRACT

An enteral feeding tube capable of delivering nutrients and/or medications to a patient at multiple points in the gastrointestinal tract and/or aspirating the gastrointestinal tract, comprising two or more channels each with at least one opening, said channels running parallel to the other until one channel ends, each channel keeping the fluid separated from the other channel, said channels being open at the proximal end and closed or open at the distal end and each channel having one or more perforations of same or variable size.

### FIELD OF THE INVENTION

The present invention provides an enteral feeding tube that has the unique ability to feed into a gastrointestinal (GI) tract of a mammal that has poor or no motility and withdraw fluid including nutrients and/or medicaments from various parts of the intestine. The invention also provides a single tube with two lumens that can pass from nasogastric route and gives ability to withdraw fluid from the stomach and at the same time feed in pat of the intestine beyond stomach. The invention further provides a feeding tube with conducting wires which aid in early detection of displacement and blockage of the tube. The conducting wires include with fibro-optic sensors.

### BACKGROUND OF THE INVENTION

Enteral nutrition is a form of feeding where the nutrients and medications are delivered directly to the gastrointestinal tract. This is the natural way of receiving nutrition and is therefore considered the ideal method of feeding. Oral feeding is the best method of delivering enteral nutrition. In patients who are unable to use the oral route of feeding for various reasons, enteral feed/fluid administration and aspiration is accomplished by use of a multitude of tubes, which may be nasogastric tube, nasojejunal tube, gastrostomy tube, gastrojejunal tube or jejunal tube; all of these are generally referred to as ‘enteral feeding tubes’.

Nasogastric and nasojejunal tubes are inserted via the nostril. The nasogastric tube passes from the nostril into the oesophagus (food pipe) and then into the stomach. In the case of the nasojejunal tube, a nasogastric tube is advanced further beyond the stomach, passing through the duodenum and into the jejunum. The advantage of nasogastric and nasojejunal tubes is that neither requires any surgical operation for their insertion. However, nasogastric and nasojejunal tubes can only be used for short duration as using these tubes for too long can lead to sinusitis, infections or ulceration of the tissue of the sinuses, throat, oesophagus or stomach. Another very important reason for these two tubes being unsuitable for long term use is they get displaced. For example, the nasogastric tube frequently gets pulled back from the stomach into the oesophagus. Similarly, the naso jejunal tube gets displaced so that the tip of the tube gets back into the stomach rather than staying in the jejunum. These displacements, if undetected can lead to serious harm to patients. For example the displacement of the nasogastric tube into oesophagus (displaced from stomach) can lead to introduction of feed into oesophagus with the serious (sometimes catastrophic) risk of feed spilling into the respiratory tract (at the top end of the gastrointestinal tract near mouth, gastrointestinal tract and respiratory tract share a common tract). Blockage of the tubes is another problem. These blockages need reinsertion if they cannot be unblocked. These reinsertions can be uncomfortable and can require reinsertion under X-ray guidance, with the risk of radiation exposure from X-rays.

Gastrostomy tubes, gastrojejunal tubes and jejunal tubes are used on a long-term basis in patients where the oral route cannot be used for enteral nutrition. Insertion of gastrostomy tubes, gastrojejunal tubes and jejunal tubes require the creation of a stoma, i.e. an artificial opening, by surgical means on the anterior abdominal wall. Gastrostomy tubes require the formation of a stoma from the stomach on the anterior abdominal wall, this procedure is called Gastrostomy. It creates a direct conduit between the stomach and abdominal wall. Through this stoma a gastrostomy tube is inserted into the stomach and feed can be given directly into the stomach through this tube. When inserting a Gastrojejunal tube, the tube is advanced through the Gastrostomy into the stomach and then advanced to the duodenum and then to jejunum. When inserting a jejunal tube, a stoma can be created from the jejunum on the anterior abdominal wall, this stoma is termed Jejunostomy. Jejunostomy is a direct conduit between the abdominal wall and lumen of the jejunum. Through a jejunostomy, a jejunal feeding tube is introduced into the jejunum for administration of feeds directly into the jejunum. Displacement of the enteral feeding tube and blockage are two common issues with these tubes, more so in gastro jejunal tubes. The gastro jejunal tubes frequently have an issue where the jejunal end of the tube gets back into the stomach from jejunum. These displacements make these tubes unsuitable for use and require reinsertion and/or replacement. These replacements and reinsertion require another operation. These displacements, if undetected can lead to serious harm to patients. For example the displacement of the gastrojejunal tube into stomach (displaced from jejunum) can lead to introduction of feed into stomach with the risk of feed refluxing back into the oesophagus, with the risk of spilling into the respiratory tract (at the top end of the gastrointestinal tract near mouth gastrointestinal tract and respiratory tract share a common tract). Blockage of the tubes can require another operation or x-ray guidance, thus exposing the patients to risk associated with another operation and radiation risk from X-rays

All the above mentioned feeding tubes (nasogastric tube, nasojejunal tube, gastrostomy tube, gastrojejunal tube and jejunal tube—generally and collectively referred to as ‘enteral feeding tubes’) require an intestinal tract that has normal motility. Intestinal motility is termed as Peristalsis. Intestinal peristalsis facilitates the forward propulsion of feed in the lumen of the intestine, enabling its absorption over the entire large surface area of the intestine.

There is a subgroup of patients which include both human and non-human mammals who have poor or no intestinal motility. This absence of motility can be a short term, medium term or long term problem. These patients cannot be fed into their intestinal tract by any of the above mentioned feeding tubes (Nasogastric tube, Nasojejunal tube, Gastrostomy tube, Gastrojejunal tube and Jejunal tube) due to the lack of peristalsis. These patients are delivered their nutrition by introducing feeds directly into their veins, and this route is called parenteral nutrition. Parenteral nutrition can be total (termed ‘Total Parenteral Nutrition’) or partial (termed ‘Partial Parenteral Nutrition’). Total parenteral nutrition (TPN) supplies all daily nutritional requirements. TPN can be used in the hospital or at home. Because TPN solutions are concentrated and can cause thrombosis of peripheral veins, a central venous catheter is usually required.

Parenteral nutrition has many disadvantages. It requires the surgical insertion of tubes/catheters/lines into the peripheral or central veins of the body. The insertion of these delivery tubes requires invasive surgical procedures with risk of serious intraoperative and post-operative complications, and they are also fraught with the risk of infection that could lead to systemic sepsis. These complications can be life threatening and fatal. Parenteral nutrition is very costly. Its safe delivery requires the availability of sophisticated sterilisation technology, advanced daily monitoring and specialised nursing care and is therefore unavailable to most of the developing world. Also, the inner lining of the intestine absorbs nutrition from the lumen of the intestine. Hence enteral nutrition is a must for the health of the inner lining of the intestine. Prolonged absence of enteral nutrition (in spite of TPN to take care of all caloric needs) could lead to atrophy of the inner lining of the intestine. This atrophy further impairs the functioning of the intestine i.e., by reducing its motility and absorption. About 5 to 10% of patients have complications related to central venous access; greater than 50% of the patients are prone to catheter-related sepsis; more than 90% of patients suffer from glucose abnormalities (hyperglycaemia or hypoglycaemia) or liver dysfunction. Further, if TPN is administered for more than 3 months then the patient is at a risk of developing metabolic bone disease or bone demineralisation (osteoporosis or osteomalacia). Advanced disease can cause severe periarticular, lower-extremity, and back pain. Additionally, limiting the mode of administration of nutrition to parenteral nutrition has adverse effects on the liver. TPN is associated with liver failure and dysfunction. By giving at least 20% of the total nutrition requirement of a person through the enteral mode of administration along with parenteral nutrition, the adverse effects on the liver can be averted. Compared with enteral nutrition, it causes more complications, does not preserve GI tract structure and function as well, and is more expensive, particularly when the use is for veterinary purposes. Thus enteral nutrition on its own or in combination with parenteral nutrition has numerous potential advantages.

The devices existing in prior art depend on the motility of the intestine to propagate the feed to the entire length of the intestine for its uniform distribution. They all deliver feed/fluid/nutrients at a single discrete point in the intestinal tract. Their designs do not allow the feed to be delivered to an intestine that has defective or dysfunctional peristalsis and/or lacks motility. It is a well-known fact that food cannot travel down the small intestine without peristalsis, which is a wavelike series of muscular contractions. It is pertinent to note that movement of feeds inside the intestine does not happen without peristalsis. Unlike material tubes/pipes where fluid can move with force of gravity or pressure, in intestines food only move forward by the coordinated movements of its wall. Thus, intestinal motility is must for the food to travel through the GI tract. This travelling of the food in the intestine is essential for the absorption of the food. Peristalsis makes the food available to entire absorptive surface of the intestine. Many patients throughout the world, especially in developing countries suffer from consequences of malnutrition and often die due to ileus (a disruption of the normal propulsive ability of the gastrointestinal tract) resulting in inadequate nutrition or other complications as aforementioned. Thus, there is a need for a device that is able to deliver nutrition at diverse and multiple points in the gastrointestinal tract of the patient regardless of the motility of the gastrointestinal tract. This enteral feeding tube can be a replacement for parenteral nutrition and additionally, may also be a valuable supplement/complement to it.

Prior art discloses several devices available for administering feed to a patient through nasogastric, nasojejunal, gastrostomy, gastrojejunal and jejunal routes.

U.S. Pat. No. 5,527,280 relates to a dual enteral feeding and medicating device for supplying nutrients/medications to a patient through a stoma penetrating into the stomach. Said feeding device comprises of an elongated jejunal feeding tube, a gastric feeding tube coaxial with said jejunal tube having a gastric lumen defined by the hollow interior of the gastric tube surrounding the jejunal tube with a distal end having an outlet within the patient's stomach and an inlet end positioned externally of the patient, a support ring mounted on the external part of said gastric tube to engage the body of the patient surrounding the external opening of the stoma, a feeding attachment at the inlet end of said gastric tube. This device has two lumens. Limitations of this device are that the feed is delivered into the jejunum at ‘one discrete point’. This device can only help patients who have intact motility (peristalsis) of their intestinal tract even then the device is rather cumbersome for use for non-human mammals who cannot understand the instructions of a healthcare worker. This device is not suitable for feeding into intestine that has poor or no peristalsis. Moreover, it does not have the capability to feed ‘directly’ into the ileum. And further, it is inserted into the patient by gastrostomy stoma, i.e., it involves surgical invasion.

U.S. Pat. No. 4,685,901 relates to a device for supplying food and medication to a patient. Said device is inserted through a stoma and into the patient's stomach for feeding into the stomach and/or the jejunum. The device claims to be capable of feeding the jejunum directly by by-passing the stomach. Limitations of this device are that the feed is delivered into the jejunum at ‘one discrete point’. This device can only help patients who have intact motility (peristalsis) in their intestinal tract. This device is not suitable for feeding into intestine that has poor or no peristalsis. Moreover, it does not have capability to feed ‘directly’ into the ileum and it is unable to simultaneously feed more than one portion of the GI tract.

U.S. Pat. No. 5,242,389 relates to a flexible, dual-lumen enteral feeding tube for delivery of fluid through a patient's oesophagus to his/her digestive system. The enteral feeding tube has a proximal and a distal end and comprises of an enteral feeding lumen having a feeding lumen inlet opening adjacent to the proximal end of the tube and a feeding lumen outlet opening adjacent the distal end of the tube. Additionally, a relatively small stylet lumen is provided separate from said enteral feeding lumen.

U.S. Pat. No. 4,594,074 relates to the administration and aspiration of fluids to and from body cavities such as the gastrointestinal tract through a catheter and, in particular, to an enteral feeding tube having a non-collapsible bolus containing a tube outlet disposed on a distal end of the tube. A distal end of the feeding tube is provided with a multiplicity of tube openings through the tube side walls which define tube outlets. Distal to the tube outlets is an elongated weighted guide tip to facilitate intubation.

Other examples of prior art enteral feeding tubes of similar design are disclosed in U.S. Pat. Nos. 4,410,320; 4,390,017; 4,270,542 and 4,388,076.

The disadvantages with the existing prior art are that they cannot optimally function in the absence of peristalsis or in the presence of defective/poor peristalsis of the intestine. By virtue of their design, all prior devices cannot efficiently provide nutrition to multiple parts of the gastro-intestinal tract. Inability to deliver some nutrition to the inner lining of the bowel that has poor or no peristalsis leads to atrophy of the lining of the small intestine and malfunctioning of the liver.

Another problem identified in the existing devices is that the tube migrates/gets displaced from its intended place in the intestine. This tendency to migrate to a wrong place in the intestine results in administration of feeds at a wrong location within the GI tract, leading to potentially disastrous consequences for the patient.

Additionally, the tubes and lumen of existing devices also have tendency to blockage which is normally detected at a delayed stage. The doctors are compelled to clear some of the blockages by flushing while other blockages may be cleared by insertion of a new tube.

The disadvantages with the existing prior art are that it is not possible to detect the migration/displacement of tube to an unintended location in the GI tract unless fluid is withdrawn from the tube and its pH is checked to ascertain whether tube is in stomach (acidic pH) or in some other part of the intestine (alkaline pH). Other way to check position of the tube is by X-ray which involves radiation. Fibro-optic sensors are sensors that use optical fibres either as the sensing elements, or as a means of relaying signals from another sensor to the electronics that process the signals.

Thus, the introduction of means such as fibro-optic sensors is desirous in determining the position of the tube. Further, these sensors are also desirous for the early detection of blockage of tube leading to quick remedial measures, avoiding tube change with more invasive means.

To overcome the drawbacks existing in the feeding devices and modes of delivery currently known and used, there exists a need to have a uniquely designed, simple and economical enteral feeding device with multiple lumens, each lumen having one or more perforations to administer and withdraw nutrients and/or medications at multiple points of the GI tract. Further, it is also desirous that such specialised and precious feeding tubes have devices such as fibre-optic sensors for early detection of migration/displacement of the tube and early detection of blockage.

These fibre-optic devices can also be useful if they are incorporated in conventional nasogastric, naso jejunal and gastro jejunal tubes. All the currently available enteral feeding tubes do not have these fibro-optic sensors. The early detection of migration/displacements of any enteral feeding tube (be it the specialised tube that can feed into bowel with poor or no peristalsis or conventional feeding tubes) can prevent accidental delivery of feed at wrong part of the intestine leading to adverse outcome (especially from spillage of feed into respiratory tract and lungs). The early detection of impending blockage can help in effective clearing of blockages. The early detection of blockage can also lead to easier replacement of the tube (on a guidewire which can pass through partially blocked tube, enabling removal of the partially blocked tube and railroading of a new tube over the guidewire). Detection of partial blockage (narrow lumen) before the blockage becomes complete (complete obliteration of the lumen is a great help in management of millions of patients on enteral feeding devices.

In summary, Such a family of devices can help millions of patients all over the world, in developed and developing countries, who have poor or no intestinal motility to absorb enteral nutrition with existing devices and suffer from the consequences of all enteral feeding tubes that get displaced and blocked.

### OBJECTS OF THE INVENTION

Accordingly, an object of the present invention is to address the existing drawbacks and administer and deliver nutrients and/or medications to patients at multiple points and/or specific points, as required, with ease, causing minimal discomfort to the patient, and to deliver nutrients and/or medications at a controllable rate with site specificity.

Another object of the present invention is to withdraw fluid including nutrients and/or medications from multiple points and/or specific points, as required, with ease, causing minimal discomfort to the patient for evaluation of digestion and/or absorption in the lumen of the intestine and/or for re-insertion/delivery though other points of the tube, of nutrients and/or medications in a site-specific manner to aid digestion and/or absorption in a different part of the intestine.

It is a further object of the present invention to provide a device with a means such as fibro-optic sensors for early detection of migration/displacement of the tube from the intended location and for early detection and treatment of blockage and prevention of complete blockage of the tube.

It is yet another object of the present invention to provide some enteral nutrition to patients on total parenteral nutrition, contributing to maintenance of superior health of the absorptive surface of the digestive tissue by keeping it in use; and contributing to superior preservation of other organs such as the liver.

### SUMMARY OF THE INVENTION

The present invention provides for a uniquely designed, simple and economical enteral feeding device with multiple lumens, each lumen having one or more perforations to administer and for withdrawing nutrients and/or medications at multiple points of the GI tract. The simplest version of this tube will be a naso-gastric-tube with two lumens, one that helps in drawing out fluid from stomach and other which can help in introduction of feeds in part of the intestine beyond stomach. The lumen to feed beyond stomach can have single opening or multiple opening.

The present invention also provides for an enteral feeding device with fibro-optic sensors for timely detection migration/displacement of the tube to an unintended location. The undetected migration leading to feed in wrong part of the intestine can lead to disastrous consequences from spillage of feed in the respiratory tract. This tube will prevent such adverse consequences of misplaced tube feeds.

The present invention also helps in detecting blockage of tube when the blockage is partial and incomplete and thus institution of early treatments to address blockages and thus preventing complete blockage. Partial blockages are much easier to treat than full complete blockage.

This family of e device of the present invention can help millions of patients all over the world, in developed and developing countries, who have poor or no intestinal motility.

Though enteral feeding tubes with multiple lumens (typically two) supplying nutrients and/or medications to different parts of the GI tract exist in the prior art, the primary drawback with the prior art devices is that they only have a single hole at their distal end. Thus they can only deliver nutrients at a single point in the intestinal tract. Therefore they are still dependent on the motility of the intestine to supply nutrients to the remaining parts of the GI tract. These devices cannot be used in feeding in an intestine that lacks motility (peristalsis). There is no multi-lumen tube that is currently available for use via nasogastric route.

These devices with one or two functional lumens have another drawback, i.e., in the event of an occlusion of a channel and inability to unblock that channel, the nutrients and/or medications may be accumulated in the tube and may not be supplied to the target organs. With the present invention, multiple lumens and multiple holes make blockages easy to manage. It is very unlikely that all channels and all holes in each channel get blocked. Moreover, the tube will still function even in the event of blockage of a few channels and/or a few holes.

In one aspect, the present invention relates to an enteral feeding tube capable of delivering nutrients and/or medications to a patient at multiple points in the gastrointestinal tract and/or aspirating the gastrointestinal tract, comprising two or more channels each with at least one opening, said channels running parallel to the other until one channel ends, each channel keeping the fluid separated from the other channel, said channels being open at the proximal end and closed or open at the distal end and each channel having one or more perforations of same or variable size.

In another aspect, the present invention relates to an enteral feeding tube capable of delivering nutrients and/or medications to a patient at multiple points in the gastrointestinal tract and/or aspirating the gastrointestinal tract, comprising two or more channels each with at least one opening, said channels running parallel to the other until one channel ends, each channel keeping the fluid separated from the other channel, said channels being open at the proximal end and closed or open at the distal end and each channel having one or more perforations of same or variable size, one or more channels having fibro-optic sensors for temperature, pH and pressure determination.

In another aspect of the present invention, the enteral feeding tube is a two lumen tube with one lumen extending till the stomach and the other lumen extending beyond the stomach.

In another embodiment, the present invention relates to an enteral feeding tube with fibro-optic sensors for light-based timely detection and prevention of migration/displacement of the tube to an unintended location.

In a further embodiment, the enteral feeding tube also has fibro-optic sensors for light-based timely detection of partial blockage and prevention of complete blockage of the tube.

In one aspect of the present invention, the enteral feeding tube is a single lumen and single hole tube having fibro-optic sensors. The fibro-optic sensors help in timely detection of migration/displacement of the tube to an unintended location and timely detection of partial blockage and prevention of complete blockage of the tube.

In another embodiment of the present invention, the lumen of the enteral feeding tube is formed by separate channels being enclosed in a common tube.

In another embodiment of the present invention, the lumen of the enteral feeding tube is formed by separate channels such that these separate channels can be split from each other without compromising the lumen of any channel. This provides the ability to tailor the length of the channel to suit patients of various heights and size.

In another embodiment of the present invention, the lumens of the enteral feeding tube are formed by creating partitions longitudinally in the tube.

In a further embodiment of the present invention, the enteral feeding device is made of any flexible and biocompatible material, such as but not limited to silicone, polyvinylchloride, polyurethane.

In another embodiment of the enteral feeding tube, the two or more channels with one or more perforations are used to withdraw fluid including nutrients and/or medication from various parts of the intestine.

In yet another embodiment of the present invention, the withdrawn fluid is evaluated for digestion or re-inserted through other perforations in other channels to aid digestion and absorption of nutrients and/or medications in different parts of the GI tract.

In another embodiment, the enteral feeding tube has conducting wires running along the entire length or part of its length.

Further, in one embodiment, the conducting wires are on the outer surface of the enteral feeding tube.

In another embodiment, the conducting wires are inside the enteral feeding tube. They can be inside the tube or in a separate channel inside the tube (this channel being separate from the main channel through which feed travels.

In another embodiment of the enteral feeding tube, the conducting wires have the ability to send electrical signals to gates regulating the opening and closing of the perforations and controlling the distribution of nutrients and/or medication in the lumen of the GI tract.

In another embodiment of the enteral feeding tube, the conducting wires have the ability to send electrical signals to stimulate movement in a localised part of the intestine to facilitate digestion and absorption of nutrients and/or medication.

In another embodiment of the enteral feeding tube, the conducting wires are fibro-optics wires that have one or more sensors to measure temperature, pressure, pH and other like parameters.

In a further embodiment of the enteral feeding tube, the conducting wires have one or more fibro-optic sensors to measure temperature, pressure and pH.

In another embodiment of the enteral feeding tube, the conducting wires have one or more fibro-optic sensors for light-based timely detection of migration/displacement of the tube to an unintended location.

In a further embodiment of the enteral feeding tube, the conducting wires have one or more fibro-optic sensors for light-based timely detection of early partial blockage and prevention of complete blockage of the tube.

In another embodiment of the present invention, the lumen of the tube is of variable internal diameters to facilitate the uniform distribution of fluids including nutrients and/or medication in the lumen of the GI tract.

In yet another embodiment of the present invention, the lumen of the tube has a solid core inside for facilitating uniform distribution of fluids including nutrients and/or medication exiting through the perforations. This solid core can be in the form of guidewire which can be inserted and pulled out of the lumen and/or channels of the tube.

In another embodiment of the present invention, the lumen of the tube has a solid core inside which runs along the entire length of the tube.

In another embodiment of the present invention, the lumen of the tube has a solid core inside which runs along the distal part of the tube.

In an embodiment of the present invention, the enteral feeding tube optionally ranges from 25 to 1000 cm in length.

In an embodiment of the present invention, length of the first channel is in the range of 5 to 50 cm and is optionally provided with one or more perforations.

In another embodiment, the length of the first channel is alternatively in the range of 10 to 50 cm and is optionally provided with one or more perforations.

In an embodiment of the present invention, the tube optionally consists of two channels with the length of second channel in the range of 25 to 1000 cm from the distal end of the first channel.

In an embodiment of the present invention, the tube optionally consists of three channels with length of second channel in the range of 25 to 1000 cm from the distal end of the first channel.

In another embodiment of the present invention, the length of the second channel is optionally in the range of 10 to 1000 cm from the distal end of the first channel.

In yet another embodiment of the present invention, the second channel has multiple perforations preferably commencing at a distance of 0 to 100 cm from the distal end of the first channel.

In yet another embodiment of the present invention, the second channel has multiple perforations optionally commencing at a distance of 5 to 50 cm from the distal end of the first channel.

In an embodiment of the present invention, channels subsequent to second channel have multiple perforations preferably commencing at a distance of 0 to 100 cm from the distal end of the second channel.

In another embodiment of the present invention, the third channel has multiple perforations optionally commencing from the distal end of the second channel.

In yet another embodiment of the present invention, the third channel has multiple perforations optionally at a distance of 0 to 100 cm from the distal end of the second channel.

Another aspect of the present invention relates to an enteral feeding tube capable of delivering nutrients and/or medications to a patient at multiple points in the gastrointestinal tract and/or aspirating the gastrointestinal tract, comprising two or more channels each with at least one opening, said channels running parallel to each other for the whole length of the tube, and ending at the same length, each channel keeping the fluid separated from the other channel, said channels being open at the proximal end and closed or open at the distal end and each channel having one or more perforations of same or variable size.

In another embodiment of the present invention, the tube has up to 100 channels and each channel has multiple perforations along its length for the exit/entry of nutrients and/or medications into the gastrointestinal tract and said perforations are of variable number, ranging from one to hundred.

Another aspect of the present invention relates to an enteral feeding tube capable of delivering nutrients and/or medications to a patient at multiple points in the gastrointestinal tract and/or aspirating the gastrointestinal tract, comprising two or more channels each with at least one opening, said channels running parallel to each other but ending at different points along the length of the tube, wherein each channel is open at both ends, each channel keeping the fluid separated from the other channel.

In one embodiment of the present invention, the tube has up to 100 channels and each channel further comprises one or more perforations along its length, wherein the perforations are of same or variable size and of variable number, ranging from one to hundred.

In another embodiment of the present invention, the channels are optionally closed at one end.

In yet another embodiment of the present invention, the enteral feeding tube is capable of being used for short or medium duration by insertion through the nasal or rectal cavity.

In a further embodiment of the present invention, said the enteral feeding tube is capable of being used for long duration by gastrostomy or jejunostomy, after modification.

Another aspect of the present invention relates to an enteral feeding tube capable of insertion in the patient through the naso-gastric route, comprising:
two or more tubes each with at least one lumen, said tubes running parallel to the other until one tube ends, each tube keeping the fluid separated from the other tube, said tubes being open at the proximal end and closed or open at the distal end and each tube other than the first tube having one or more holes of same or variable size,
a lumen each for stomach and jejunum and ileum of small intestine,
common tube enclosing the channels,
the first opening in the first channel for the stomach, and
the second set of openings for the small intestine, including multiple holes along the length of the channel, which start 1-50 cm from the end of the channel ending in the stomach. In addition to these openings, there are one or more openings towards the distal end of this channel.
These devices can be inserted into the gastrointestinal tract using a guide wire through the gastric lumen,
Or through using a guide wire through the jejuno-ileal lumen.

A further aspect of the present invention relates to an enteral feeding tube capable of insertion by gastrostomy or jejunostomy, comprising:
two or more tubes each with at least one lumen, said tubes running parallel to the other until one tube ends, each tube keeping the fluid separated from the other tube, said tubes being open at the proximal end and closed or open at the distal end and each tube other than the first tube having one or more perforations of same or variable size,
a lumen each for stomach and jejunum and ileum of small intestine,
common channel enclosing the tubes,
an opening in the first lumen for the stomach,
second lumen for small bowel not perforated at the proximal end,
a guide wire through the gastric lumen,
a guide wire through the jejuno-ileal lumen,
a ring,
a hollow tube, and
a disc shaped structure.

In another embodiment of the present invention, the ring is capable of sliding freely on the enteral feeding tube to keep the tube in position outside the body on the abdominal wall.

In yet another embodiment of the present invention, the hollow tube comprises of a hole which is perpendicular to the lumen of the hollow tube, through which the enteral feeding tube passes.

In a further embodiment of the present invention, the hollow tube is capable of sliding freely on the enteral feeding tube.

In yet another embodiment of the present invention, the disc shaped structure, fixed to the tube, is positioned inside the gastrointestinal tract and also outside the abdominal wall.

In yet another embodiment of the present invention, the disc shaped structure along with the sliding ring and hollow tube enable fixing of the tube in gastrostomy or jejunostomy insertions.

In a further embodiment of the present invention, the enteral feeding tube is capable of being shortened by cutting at proximal or/and distal end to tailor its length to fit patients of various sizes and builds.

In yet another embodiment of the present invention, an enteral feeding tube capable of insertion in the patient through the naso-gastric route is provided. The tube comprises two lumens wherein two lumens run parallel to each other (9) wherein one lumen opens into the stomach (33) with a single opening (11) and the second lumen goes beyond the outlet of the stomach into the duodenum (34) or jejunum (35) and has no side hole or multiple side holes, but has a hole near its end (52).

### BRIEF DESCRIPTION OF THE ACCOMPANYING DRAWINGS

Embodiments of the present invention are illustrated by way of example and not by way of limitation in the figures of the accompanying drawings, in which like references indicate similar elements and in which:

FIG. 1: Short-length enteral feeding tube with two channels.

FIG. 2: Cross-sectional view at point 9 of FIG. 1.

FIG. 3: Cross sectional view at point 13 of FIG. 1.

FIG. 4: Cross sectional view at point 15 of FIG. 1. It shows one of the many holes (23) in the second channel for the exit of nutrients/medications.

FIG. 5: Long-length enteral feeding tube with two channels for insertion through nasogastric route.

FIG. 6: Long-length enteral feeding tube with two channels for insertion through gastrostomy.

FIG. 7: Long-length enteral feeding tube with three channels for insertion through nasogastric route.

FIG. 8: Long-length enteral feeding tube with three channels for insertion through gastrostomy.

FIG. 9: Long-length enteral feeding tube with multiple channels (27) (up to 100 channels) for insertion through nasogastric route, gastrostomy, jejunostomy or rectal route with channels running parallel to each other for the entire length of the tube.

FIG. 10: Long-length enteral feeding tube with multiple channels (27) (up to 100 channels) for insertion through nasogastric route, gastrostomy, jejunostomy or rectal route with channels parallel to each other for variable length of tube and each channel with no holes.

FIG. 11: Long-length enteral feeding tube with multiple channels (27) (up to 100 channels) for insertion through nasogastric route, gastrostomy, jejunostomy or rectal route with channels parallel to each other for variable length of tube and each channel with multiple side holes.

FIG. 12: Cross-sectional view at point 27 of FIGS. 10, 11.

FIG. 13: Cross-sectional view at point 26 of FIG. 10.

FIG. 14: Long-length enteral feeding device with two channels, inserted through the nasogastric route.

FIG. 15: Long-length enteral feeding device with two channels, inserted through the gastrostomy route

FIG. 16: Short-length enteral feeding device with two channels, inserted through the nasogastric route

FIG. 17: Enteral Feeding device with conducting wire (40) as sensor/signaller. The conducting wire (40) is incorporated inside one of the lumens of the enteral feeding device.

FIG. 18: Enteral Feeding device with conducting wire (40) as sensor/signaller. The conducting wire (40) is incorporated outside on the wall of the enteral feeding device.

FIG. 19: Enteral Feeding device with conducting wire (40) as sensor/signaller. The conducting wire (40) is incorporated in the outside wall of the tube.

FIG. 20: Enteral Feeding device with conducting wire (40) as sensor/signaller. The conducting wire (40) is incorporated inside on the wall of the enteral feeding device.

FIG. 21: Enteral feeding device (shown in longitudinal section) with solid core (44) in the lumen of the tube (24).

FIG. 22: Enteral feeding device (shown in cross section) with solid core (44) in the lumen of the tube (24).

FIG. 23: Enteral feeding device with variable lumen size and wall thickness (longitudinal section).

FIG. 24: Enteral feeding device with variable lumen size and wall thickness (cross section at the narrow lumen). The thickened wall (47) with a narrow lumen (45) and hole (23) in the wall.

FIG. 25: Enteral feeding device with variable lumen size and wall thickness (cross section at the wider lumen). The thin wall (47) with a broader lumen (46) and hole (23) in the wall.

FIG. 26: Nasogastric tube with fibro-optic sensor—the tube in proper position.

FIG. 27: Nasogastric tube with fibro-optic sensors—the tube is in improper position as it has slipped from the stomach into the oesophagus.

FIG. 28: Nasogastro-duodenal tube with fibro-optic sensors—the tube is in proper position.

FIG. 29: Nasogastro-duodenal tube with fibro-optic sensors—the tube is in improper position as it has slipped back from duodenum into the stomach.

FIG. 30: Gastroduodenal tube with fibro-optic sensors—the tube is in proper position.

FIG. 31: Gastroduodenal tube with fibro-optic sensors. The tube is in improper position—the gastroduodenal tube has migrated so that the tip of the tube lies in the stomach instead of the duodenum—which is the intended place for delivery of feeds.

FIG. 32: Longitudinal section of the feeding tube with fibro-optic sensors—dual lumen.

FIG. 33: Longitudinal section of the feeding tube with fibro-optic sensors—three lumen.

FIG. 34: Cross section of the enteral feeding tube with fibro-optic sensors

FIG. 35: Cross section of enteral feeding tube with two fibro-optic sensors.

FIG. 36: The enteral feeding tube with double lumen for nasogastric route.

### KEY FOR LABELS ON DRAWINGS

| Reference | |
|---|---|
| Numerals | Label |
| 1 | Guide wire through the Gastric lumen of the enteral feeding tube |
| 2 | Guide wire through the Jejunal lumen of the enteral feeding tube |
| 3 | Guide wire through the Ileal lumen of the enteral feeding tube |
| 4 | Guide wire through the jejuno-ileal lumen |
| 5 | Gastric Lumen of the enteral feeding tube |
| 6 | Jejunal Lumen of the enteral feeding tube |
| 7 | Ileal Lumen |
| 8 | Lumen for small bowel (jejunum and ileum) |
| 9 | Common channel of the tube (it will have 2 parallel equal size lumens) |
| | inside |
| 10 | Common channel of the tube (three parallel equal size lumens) |
| 11 | Lumen for stomach, opening into stomach |
| 12 | Tube with two lumens internally, one for jejunum and other for ileum (this |
| | part has no holes) |
| 13 | Tube with one lumen only for small bowel (this part of the tube has no holes) |
| 14 | Part of the tube in which has two lumens-the lumen for jejunum has |
| | multiple holes, the lumen for ileum has no holes |
| 15 | Part of the tube that has one lumen for small bowel and this has multiple |
| | holes all around its circumference |
| 16 | Lumen for jejunum ends blindly |
| 17 | Part of the tube in which the lumen for ileum has multiple holes |
| 18 | Lumen for ileum ends blindly |
| 19 | Lumen for small bowel ends blindly |
| 20 | Ring to keep the tube in position outside the body on the abdominal wall. |
| | This ring can slide freely on the enteral feeding tube |
| 21 | T bar (hollow tube) with a hole (perpendicular to the lumen of the T bar) |
| | through which the enteral feeding tube passes. This T bar can slide freely on |
| | the enteral feeding tube. |
| 22 | Disc shaped structure fixed to the tube. This will sit inside the wall of the |
| | stomach and along with the sliding ring and T bar will help to fix the tube in |
| | Gastrostomy or Jejunostomy version of the tube |
| 23 | Holes in the lumen of the tube |
| 24 | Lumen of the tube |
| 25 | Guide wire |
| 26 | Side hole |
| 27 | Multiple channels |
| 28 | Individual channel on way to be connected to pump |
| 29 | Individual channel ending blindly |
| 30 | Individual channel ending as hole |
| 31 | Nasal cavity |
| 32 | Oesophagus |
| 33 | Stomach |
| 34 | Duodenum |
| 35 | Jejunum |
| 36 | Ileum |
| 37 | Appendix |
| 38 | Large intestine |
| 39 | Rectum opening to outside through rectal cavity |
| 40 | Conducting wire which can act as Sensor and signaller |
| 41 | Connectors connecting the conducting wire to the gates controlling the |
| | openings in the device/channel of the device |
| 42 | Gate controlling the openings in the device/channel of the device-gate is |
| | open and allows the contents to move from the lumen of the tube/channel to |
| | outside (into the lumen of the intestine). |
| 43 | Gate controlling the openings in the device/channel of the device-gate is |
| | closed and prevents the contents to move from the lumen of the tube/channel |
| | to outside (into the lumen of the intestine). |
| 44 | Solid core inside the lumen of the tube or channel. This solid core helps in |
| | concentric flow of the fluids in the lumen of the tube. |
| 45 | Part of the tube which has a narrower lumen and thicker wall |
| 46 | Part of the tube which has wider lumen and thinner wall |
| 47 | Wall of the tube-it can have variable thickness giving rise to variable sized |
| | lumen (on the outer side the tube has uniform diameter) |
| 48 | Double lumen feeding tube, one lumen for feeds and other lumen for fibro- |
| | optic sensor wire |
| 49 | Fibro-optic sensor wire |
| 50 | Single hole in nasogastric tube |
| 51 | Single or double lumen gastroduodenal or gastro-jejeunal tube. One lumen |
| | opens into stomach and other lumen opens into duodenum or jejunum. Both |
| | lumens can have fibro-optic sensors. |
| 52 | Duodenal opening of the gastroduodenal tube |
| 53 | lumen for feeds in the double lumen and three lumen feeding tube |
| 54 | lumen for fibro-optic sensor wire in the double lumen and three lumen |
| | feeding tube |
| 55 | Part of the tube that goes beyond stomach into duodenum. This part can have |
| | an end hole or multiple holes in part of the tube beyond the outlet of the |
| | stomach. |

Descriptions of certain details and implementations follow, including a description of the figures, which may depict some or all of the embodiments or implementations of the inventive concepts presented herein. An overview of embodiments of the invention is provided below, followed by a more detailed description with reference to the figures.

### DETAILED DESCRIPTION OF THE INVENTION

The present invention relates to a uniquely designed, simple and economical enteral feeding device for administering and withdrawing nutrients and medication from an intestine with poor or no motility. It also has the fibro-optic sensors to facilitate early migration of tube to unintended locations in the gastrointestinal tract and helping in early detection of partial blockages (thus helping in their early treatment and thus preventing complete blockage).

The enteral feeding device of the present invention overcomes the drawbacks in prior art by providing enteral feeding tube with multiple channels, each channel with multiple perforations, thereby allowing the nutrients and/or medications to be supplied to multiple discrete points throughout the entire gastrointestinal tract that has poor or no motility or peristalsis. The said design of multiple channels with multiple perforations also enables fluid including nutrients and/or medications to be withdrawn from various discrete points of the entire gastrointestinal tract having poor or no motility or peristalsis. The withdrawn fluid may be used for evaluating the digestion and/or absorption in the lumen of the GI tract or may be re-inserted through different channels and different perforations into different portions of the GI tract to aid the digestion and/or absorption of nutrients and medications in the fluid.

Further, there exists two main problem in various versions of the existing enteral feeding tubes (with single opening and single or double lumen) such as naso-gastric tubes (single lumen and single distal opening) and gastro-jejunal tubes (double lumen but single distal opening in each channel/lumen): 1) of migration/displacement of the tube to an unintended location in normal feeding scenarios 2) blockage of tube or channels. The introduction of fibro-optic channels in the tube being described in this application addresses' the above mentioned two problems. These sensors can be very useful once introduced in the singe lumen nasogastric and double lumen gastro-jejunal tubes to address the two issues of: 1) Migration—leading to introduction of feed at unintended part of the intestine that can lead to disastrous consequences and 2) Detection of partial blockage of tubes/channels—leading to introduction of early treatment prevent complete blockage and the consequences from complete blockage.

Nasogastric tubes are very common mode of feeding into the stomach in patients who cannot tolerate oral feeds. The feeds are given in the form of bolus feeds at 2-4 hours' intervals. Before administration of bolus feed the position of the feed is checked by aspirating the enteral contents and checking their pH. The acidic pH suggests that the tip of the tube is in stomach and feed can be given safely. In night, the bolus feed has a disadvantage as it needs a health-care professional to check pH of the fluid withdrawn from the nasogastric tube 3-4 times/night shift. This is plausible in a hospital setting but is difficult in a home setting. A continuous feed delivered by a pump overnight saves multiple checking of pH in the night. However, the disadvantage of continuous feed is that a nasogastric tube can slip into oesophagus and in the absence of proper pH checking (to check the position of the tube, ensuring that the tip of the tube is in the stomach), the feed can be delivered into oesophagus with risk of its aspiration into lungs. The nasogastric tube with fibro-optic sensors ensures that the feed is safely delivered into the stomach in patients who require continuous feeds into stomach without the risk of the feed accidently being given into the oesophagus (food pipe) from accidental slippage of the tube from the stomach into the oesophagus. Thus continuous feeds can be given with this tube with fibro-optic sensors at home leading to treatment of all these patients at home (more friendly setting) rather than in hospital (cost and risk of hospital acquired infections).

This is achieved by the design of the present invention that enables incorporation of fibro-optic sensors in the wall of the tube that will monitor acidity (pH) of the area around the exit site of the tube. The slippage of tube from stomach into oesophagus will activate sensors that pH is alkaline and raise alarm so that feeds can be stopped (manually or automatically), thus preventing accidental administration of the feed in the food pipe and aspiration consequences.

The fibro-optic sensors of the present invention can measure pH, pressure, curvature and temperature in various parts of the intestine especially where feed is delivered. These sensors and various measurements by them can help in determining the position of the tip of the tube and study the microenvironment of the intestine in its various parts. The position of the tip of the tube is a vital piece of information as all these tubes have tendency to slip into unintended parts of the intestine, leading to administration of feeds at wrong location in intestine which can result in serious, potentially fatal consequences.

The tubes and their various lumens also have tendency to get blocked, some of the blockages can be cleared by flushing while others need insertion of a new tube. The pressure and temperature sensors can give an early indication of blockage leading to early and more aggressive flushing, thus preventing complete blockage and reducing the incidence of new tube exchanges. Thus, these enteral feeding tubes with fibro-optic sensors address two main issues with various enteral feeding tubes such as nasogastric, nasojejunal, gastrostomy, gastrojejunal namely: 1) Migration and 2) Blockage.

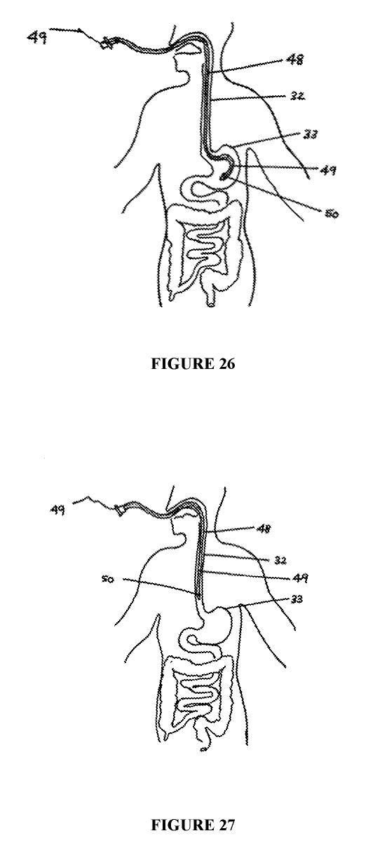

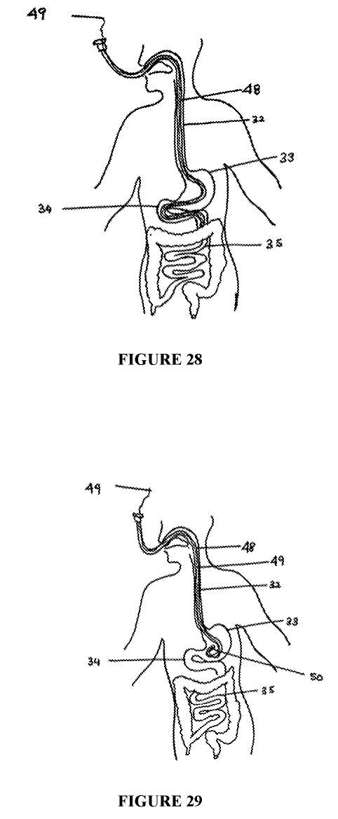

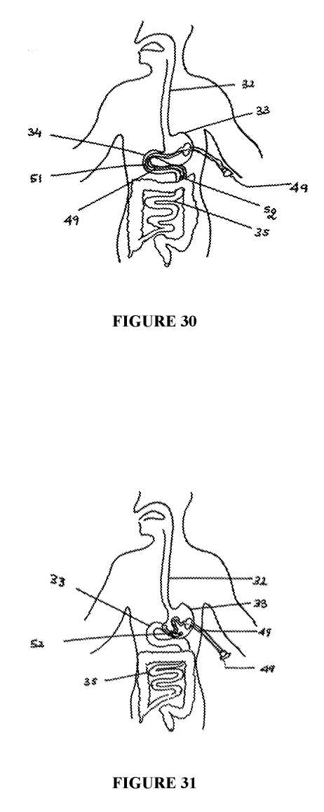

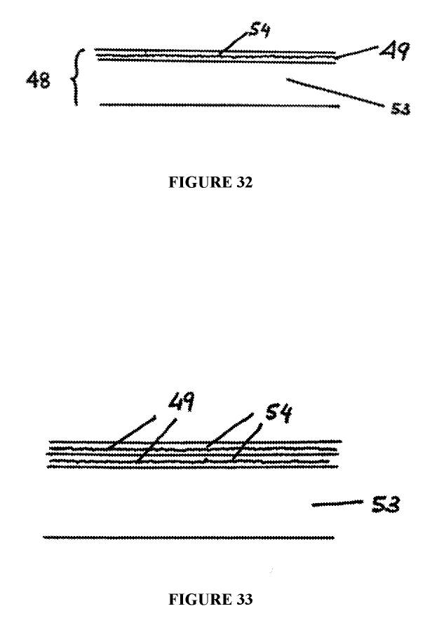

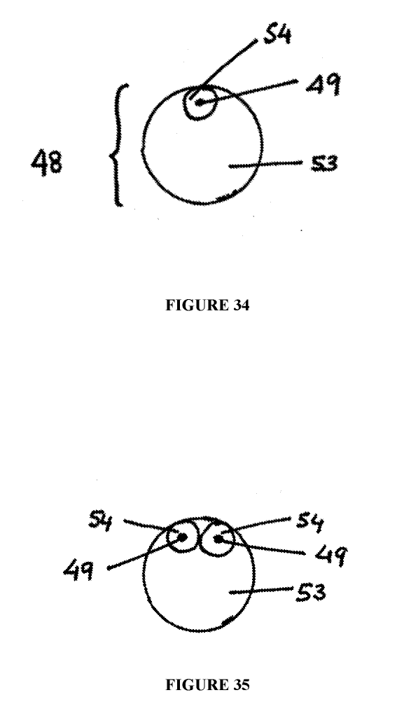

Illustrative figures demonstrating the enteral feeding tube with fibro-optic sensors in operation are described herein below:
FIG. 26 shows nasogastric tube (48) with fibro-optic sensor (49) with the tube (48) in proper position. The tube (48) has its tip in the stomach (33). The sensor (49) shows an acidic pH, confirming that the tip of the nasogastric tube (48) is in the stomach (33) and the feed is being introduced into the lumen of the stomach (33) (the intended place for the delivery of the feed).
FIG. 27 demonstrates the nasogastric tube (48) with fibro-optic sensors (49) with the tube (48) in improper position having slipped from the intended target i.e., stomach (33) into the oesophagus (32). The fibro-optic sensor (49) placed in the nasogastric tube (48) reveals an alkaline pH (instead of the acidic pH, if the tip of the tube is in the lumen of the stomach (33) i.e. the intended place for the delivery of the nasogastric feed). The alkaline pH warns that tube is in oesophagus (32)—a wrong place for the feed to be instilled and the sensor initiates a feedback mechanism alerting the care giver administering the enteral feeding device. Feed given into the oesophagus (32) can spill into the lungs with disastrous consequences for the patient.
FIG. 28 shows the nasogastro-duodenal tube (48) with fibro-optic sensors (49) with the tube (48) in proper position. The tube (48) passes through the oesophagus (32), stomach (33) into the duodenum (34) (can also go further into the jejunum (35) (called naso-jejunal tube). The purpose of this tube is to instill the enteral feed into duodenum (34) (in case of naso-duodenal tube) or jejunum (35) (in case of naso jejunal tube). The fibro-optic sensors (49) in the tube (48) reveal pH as alkaline if the tube is in duodenum (34) or jejunum (35). The alkaline pH reassures that feed is being delivered beyond the stomach (33) into duodenum (34) or jejunum (35) (indented place for the feed).
FIG. 29 illustrates Nasogastro-duodenal tube (48) with fibro-optic sensors (49) with the tube (48) in improper position as it has slipped back from duodenum (34) into the stomach (33). The tube has migrated back into the stomach (33) from the duodenum (34) (in naso-duodenal tube) or jejunum (35) (in naso jejunal tube). The purpose of this tube is to instill the enteral feed into duodenum (34) (in case of naso-duodenal tube) or jejunum (35) (in case of naso-jejunal tube). The fibro-optic sensors (49) in the tube reveal an acidic pH suggesting that the tube has its tip in the stomach (33). This warns the clinicians and carers to stop the feeds, as the feed in the stomach (33) can reflux into oesophagus (32) with the risk of spillage into lungs (with disastrous consequences) in patients with severe gastro-oesophageal reflux. Gastroduodenal tube is indicated in such patients to prevent gastro-oesophageal reflux of feed into oesophagus from the stomach.
FIG. 30 shows a Gastroduodenal tube (51) with fibro-optic sensors (49) with the tube being in proper position. This tube (51) is inserted via gastrostomy (an operation where the tube is inserted through the abdominal wall into the stomach (33) and further advanced from the stomach (33) into duodenum (34). The fibro-optic sensors (49) in the tube show an alkaline pH, confirming its position in the duodenum (34) (intended position for the delivery of the feed into duodenum(34)). The alkaline pH reassures that feed is being delivered beyond the stomach (33) into duodenum (34) or jejeunum. (35).
FIG. 31 shows a Gastroduodenal tube with fibro-optic sensors (49). The tube is in improper position—the gastroduodenal tube has migrated so that the tip of the tube lies in the stomach instead of the duodenum—which is the intended place for delivery of feeds. The fibro-optic sensors show an acidic pH, confirming the position of the gastro-duodenal tube's tip in the stomach. This warns the clinicians and carers to stop the feeds, as the feed in the stomach can reflux into oesophagus (32) with the risk of spillage into lungs (with disastrous, potentially fatal consequences) in patients with severe gastro-oesophageal reflux. Gastroduodenal tube is indicated in such patients to prevent gastro-oesophageal reflux of feed into oesophagus from the stomach.
   Illustrative figures showing longitudinal and cross-sections of the enteral feeding tube with fibro-optic sensors are explained herein below:
FIG. 32 illustrates a longitudinal section of the feeding tube (48) with fibro-optic sensors (49). The enteral tube (48) is dual lumen with one bigger lumen (53) for feeds and a smaller lumen for the sensor (54).
FIG. 33 shows a longitudinal section of the feeding tube with fibro-optic sensors (49). The enteral tube has three lumens with one bigger lumen (53) for feeds and two smaller lumens (54) for the two fibro-optic sensors.
FIG. 34 shows a cross section of the enteral feeding tube (48) with fibro-optic sensors (49). The enteral tube is dual lumen with one bigger lumen (53) for feeds and a smaller lumen (54) for the sensor.
FIG. 35 shows a cross section of enteral feeding tube with two fibro-optic sensors. The enteral tube has three lumens with one bigger lumen (53) for feeds and two smaller lumens for the two fibro-optic sensors (54).
   Thus, from the above disclosure, it is clear that the present invention provides for fibro-optic sensors for enabling timely detection of migration and early detection of partial blockage in the enteral feeding tube.

The multiple delivery points of the tube enable the simultaneous delivery of nutrients and medications to specific multiple target areas of the GI tract. It allows early introduction of enteral feed in post-operative patients who cannot be fed with existing enteral feeding devices (due to lack of intestinal peristalsis) contributing to their speedy recovery, especially those who are malnourished, postoperative or ICU patients. This enteral feeding tube can be a lifeline for the patients who have permanent loss of intestinal motility from various congenital and acquired causes. It can be an alternative or complement for parenteral nutrition, minimising the side effects of parenteral nutrition. This invention can be used to save lives in the developing world, where TPN is very costly and is not frequently used due to its sophisticated procedure and the care and monitoring involved, whereas the present invention is economical, simple to insert and easy to maintain.

Multiple perforations in the tubes of the enteral feeding device allow delivery of nutrition at multiple discrete points (up to hundreds) in the digestive system, leading to absorption of nutrients at those points. This allows feed to be given in an intestine which has dysfunctional, defective or weak peristalsis. Thus, the need for parenteral nutrition (intravenous nutrition) and associated costs and associated complications are minimised.

The gastrointestinal tract absorbs different nutrients in different sections. For instance, carbohydrates are absorbed predominantly in the jejunum i.e., the proximal part of the intestine while fats are absorbed in the ileum i.e., distal part of the small intestine. The present invention has a tube with multiple channels and each channel can have multiple perforations that allow simultaneous feeding of different fluids into different parts of the digestive tract. Thus it facilitates introduction of specialized feeds for different parts of the digestive system. It also enables draining of secretions of certain parts of the digestive tract such as the stomach.

The present invention provides a tube which can have multiple channels (ranging from 2 to 100), each channel having multiple perforations (ranging from 1 to 100) in order to facilitate delivery of nutrients and/or medications to various parts of the gastrointestinal tract, including the stomach; the duodenum, the jejunum and the ileum of patients who have an inability to eat orally and cannot be fed into their stomach or intestinal tract with existing enteral feeding tubes due to impaired or absent intestinal motility (peristalsis). The tubes described in the current invention serve as a substitute or complement to parenteral nutrition.

There are various versions of the tube that can be inserted through the naso-gastric route for short term use or through a Gastrostomy or Jejunostomy for long-term use. Short-term use is important in patients who lack intestinal peristalsis for a period of days to weeks. All patients who have complex abdominal operations leading to temporary paralysis of the intestine may benefit from early introduction of enteral nutrition (when the intestine is still in paralysis or recovering from paralysis) by these tubes leading to speedy post-operative recovery. Thus, this invention addresses many common problems due to lack of nutrition faced post-surgery, particularly in abdominal surgery in millions of patients worldwide. Long-term use can be life-saving in patients who have congenital or acquired permanent loss of intestinal peristalsis from various causes.

Said device may be a long length enteral feeding device or short length enteral feeding device, depending on the length of the tubes, duration and purpose of use of the device. The long-length device allows nutrition to be delivered to various points in almost the entire small intestine, usually on a long-term basis, overcoming the drawback of other modes of administration of nutrients to patients with impaired peristalsis, mainly parenteral (intravenous) nutrition. The short-length device allows nutrition to be delivered into the proximal part of small intestine, usually on short term basis, beyond the stomach while the stomach is in paralytic ileus and not able to receive nutrition.

The functions of short length and long length tubes are different, but they share many common structural features. The lumen in both the tubes is made of multiple and separate channels. These multiple and separate channels can be made by joining multiple single lumen tubes or by creating partitions longitudinally in the lumen of the main tube. The lengths and diameters (overall and of the individual channels) of the tube are variable and depend on the weight, height, age and condition of the patient, be it an infant or an extremely aged person. These channels can be left open at either end or closed at one end. This can help in tailoring of the tube by cutting at proximal and/or distal end to suit the physical dimensions of various lengths of the intestine in various patients. The device is made up of a material that has flexibility and biocompatibility such as but not limited to silicone, polyvinylchloride, polyurethane.

### Short Enteral Feeding Tube

After any abdominal operation, especially one which involves the handling of intestines, the entire bowel (including stomach, small intestine and large intestine (38)) suffers from paralytic ileus. The bowel does not have any motility while it is in paralytic ileus.

In normal circumstances, nutrients move in the gastrointestinal tract from stomach to small intestine to large intestine due to coordinated movements of the intestinal wall termed peristalsis. As nutritional matter moves along the gastrointestinal tract it gets digested and absorbed over its entire surface area spread over its length and breadth. Paralytic ileus is characterized by the absence of peristalsis. In this state, it is not possible to feed the patient orally or by existing enteral feeding tubes; as there is no intestinal motility to propagate the feed anywhere in the intestine for its absorption. In paralytic ileus the feed delivered at a single point, with currently existing devices, accumulates as a pool near the point of delivery. Such delivered feed fails to move forward in the intestine and therefore is unable to be adequately absorbed. Intestines behave unlike non-living tubes made of man-made material where feed introduced at one end can flow along the pressure gradient. The existing enteral feeding tube and devices depend on the intestinal motility for uniform distribution of feeds in the intestine. Paralytic ileus following an abdominal operation can take a few days or weeks to be reversed, recovery period depending on condition and the underlying pathology.

The small intestine is the first to recover from paralytic ileus. This recovery can be detected by the presence of bowel sounds that can be heard on auscultation of the abdomen by a stethoscope. The recovery of the small intestine is followed by recovery of the large intestine, which is detected by passage of flatus through the anus. The recovery of the large intestine is followed by the recovery of the stomach, which is signified by clearing of the nasogastric aspirates (they become non-bilious, in other words, clearer in colour).

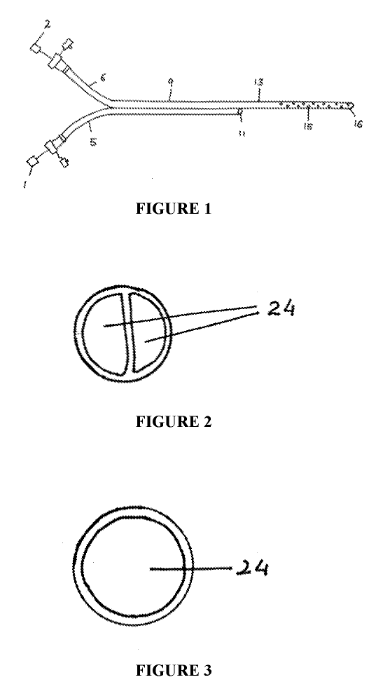

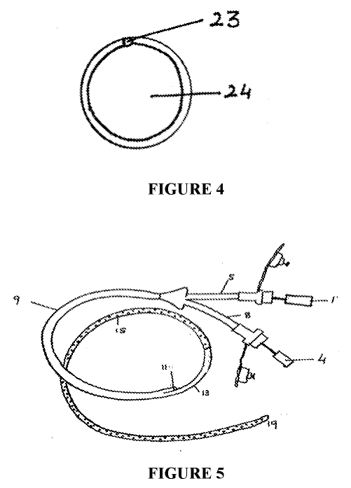

The patients can be fed orally or through the nasogastric route only after the recovery of the stomach. Since the small intestine recovers before the recovery of stomach, it is possible to feed into the jejunum while the stomach is still in paralytic ileus. In order to accomplish this, one needs access to both the stomach and the jejunum simultaneously. If access to both the stomach and the jejunum is possible at the same time by a single tube, it is possible to aspirate the stomach contents (as treatment of stomach that is in paralytic ileus and unable to accept feed) and feed into the small intestine through the jejunum (while the jejunum is recovering or has recovered from the paralytic ileus and ready to accept feeds). This is possible with short enteral feeding tube by virtue of its design, i.e. two channels in a single tube, one channel for aspirating the stomach and other channel to introduce feed into jejunum. The channel for jejunum has multiple perforations that facilitate early introduction of feed into jejunum that could still be recovering from paralytic ileus. The short enteral feeding tube can facilitate early introduction of enteral nutrition in all patients recovering from major abdominal operation. Thus short enteral feeding tube will help to establish enteral nutrition earlier than possible as compared to the devices and state of practice currently known. Early nutrition will have immense benefits in all patients especially those who are malnourished and under-nourished before any operation (a common scenario in emergency operations). In a post-surgical event, especially in case of abdominal operations, medicines are required to be delivered to a particular site in order to enable them to act and be bio-available. The present invention enables medicines to be administered to the stomach and different parts of the small intestine simultaneously and may be inserted through the nasal or rectal route, as required. The short-length device of the present invention is further illustrated in FIGS. 1 to 4 and FIG. 16.
FIG. 1 illustrates a short-length enteral feeding tube with two channels wherein the first channel has one hole at the distal end. This channel can also be closed at the end with a side hole near the end. The second channel is closed at the end but can also be left open. The second channel has multiple perforations.
FIGS. 2 to 4 show cross-sectional views at various operational points of the short-length enteral feeding device with FIG. 4 showing one of the many holes (23) in the second channel for the exit of nutrients/medications.

The short-length device has a capacity to traverse the upper gastrointestinal tract i.e., naso pharynx, oropharynx, oesophagus, stomach and the proximal part of jejunum. Said tube has at least one lumen wherein the first lumen has a solitary opening at its distal end while the subsequent lumens have multiple perforations at various points throughout their lengths. The lumens with multiple perforations may be closed at the distal end in some versions and open in other versions. In some versions these perforations may be of the same size, while in some versions they can be of variable sizes, for example smaller in size towards the proximal end and bigger in size towards the distal end or vice versa. Said tube can be inserted by nasogastric route.

FIG. 16 illustrates a version of the short-length enteral feeding device with two channels which can be inserted by nasogastric route.

According to another embodiment of the present invention, the short-length device of the present invention comprises two or more channels. All the channels are open at the proximal end and may be closed or open at the distal end. Said channels run parallel/side by side to each other until one channel ends. Also, each channel keeps the fluid separated from the other channel. The first channel has a preferred length ranging from 10 to 50 cm. This channel has one or multiple perforations along the length of the channel. Further, the second channel preferably continues a further 10 to 500 cm from the distal end of the first channel. This channel can have multiple perforations, ranging into more than hundred, along its length after the first channel ends. In a preferred embodiment these multiple perforations commence 5 to 25 cm after the point at which the first channel ends. These perforations can be of the same size or of variable size. If they are of variable size, they can have their size decreasing or increasing from the proximal to distal side of the channel.

### Long Length Enteral Feeding Tube

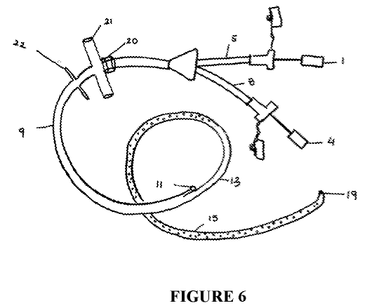

To address the need for feeding at various points in the entire digestive system for a long duration of time ranging into months and maybe years, the long-length device of the present invention provides features including a long tube that can traverse the entire gastrointestinal tract, i.e. the naso-pharynx, oropharynx, oesophagus, stomach, jejunum and ileum. Said tube has multiple channels, wherein first channel has an opening at its distal end and may have one or more openings along its length, while the subsequent channels can have one or multiple perforations at various points throughout their length. The channels with multiple perforations may be closed at the end in some versions and open in other versions. In some versions these perforations can be of the same size, while in other versions they can be of variable size, for example they may be smaller in size towards the proximal end and bigger in size towards the distal end or vice versa. Said tube can be inserted by naso-gastric route or through gastrostomy or jejunostomy or per rectal route.
FIG. 5 illustrates long-length enteral feeding tube with two channels for insertion through nasogastric route. The first channel has a hole at the distal end. This channel can also be closed at the end with a side hole near the end. The second channel is closed at the end but can also be left open. The second channel has multiple perforations.
FIG. 6 shows Long-length enteral feeding tube with two channels for insertion through gastrostomy. The first channel has a hole at the distal end. This channel can also be closed at the end with a side hole near the end. The second channel is closed at the end but can also be left open. The second channel has multiple perforations.

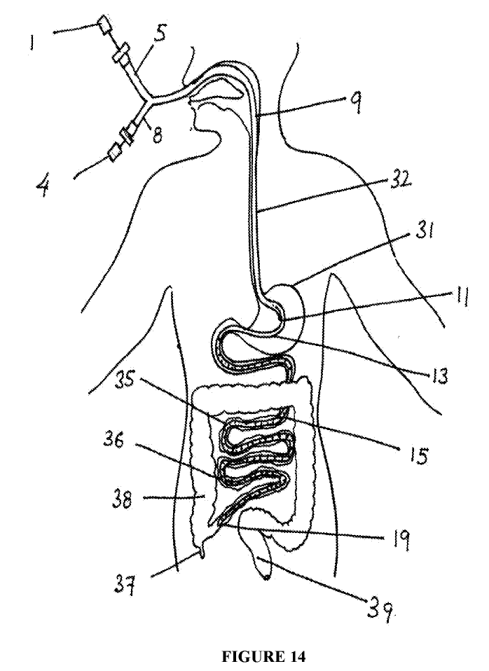

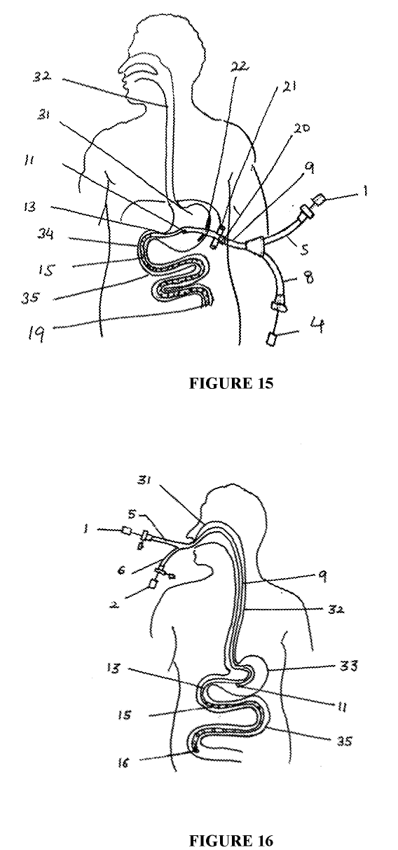

The long length of the present device thereby allows the entire intestinal tract from stomach, duodenum, jejunum to ileum to be accessible for the delivery of nutrients as shown in FIGS. 14 and 15. The multiple perforations along the length of the tubes subsequent to the first channel allow delivery of nutrition at multiple discrete points (up to hundreds) in the digestive system, therefore leading to absorption of nutrition at those points. This allows delivery of nutrition into an intestine which lacks peristaltic movement, thereby negating or minimizing many complications that may arise from feeding by parenteral nutrition (the method currently in use) such as infection, sepsis and liver failure.

The Long Enteral Feeding Tube can have number of channels ranging from 2 to 100. There are four main versions depending on the number of channels:
1) One Channel Tube
2) Two Channel Tube
1) Three Channel Tube
2) Multiple Channel Tube (number of channels from 4 to 100)
   These tubes can be inserted via any of the following routes:
1) Nasogastric Route
2) Gastrostomy Route
3) Jejunostomy Route
4) Rectal Route

According to another embodiment, channels subsequent to the second channel have the same length beyond the distal end of the previous channel as described for the previous channels. The only difference in the relationship is that the perforations may commence from the distal end of the previous channel itself.

According to another embodiment of the present invention, the long-length version of the device comprising two or more lumens, preferably in the range of 50 to 1000 cm in length. Each channel is open at the proximal end and may be closed or open at the distal end. Said channels run parallel to each other until one channel ends. Each channel keeps fluid separated from the other channel.

According to a preferred embodiment, the first channel has a length ranging from 5 to 50 cm with one or more perforations at the distal end.

According to another embodiment of the present invention, the enteral feeding device is in the form of a two-channel version, as illustrated in FIGS. 5 and 6, wherein the second channel preferably ranges from 50 to 450 cm in length from the distal end of the first channel. The second channel has multiple perforations along its length, more than a few hundred, after the distal end of the first channel. These multiple perforations preferably commence from 5 to 75 cm from the distal end of the first channel. These perforations may be of the same size or variable size. If they are of variable size, their size may increase or decrease from the proximal to the distal end of the second channel.

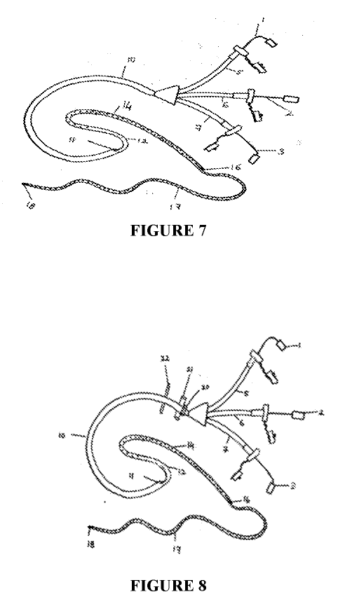

According to a further embodiment of the present invention, the enteral feeding device is in the form of a three-channel version, as illustrated in FIGS. 7 and 8, wherein the second channel preferably ranges from 50 to 250 cm in length from the distal end of the first channel. While FIG. 7 illustrates Long-length enteral feeding tube with three channels for insertion through nasogastric route, FIG. 8 shows the said tube for insertion though gastrostomy. In both these illustrations, the first channel has end hole. This channel can also be closed at the end with a side hole near the end. The second channel is closed at the end but can also be left open. The second channel has multiple holes/perforations. The third channel is closed at the end but can also be left open. The third channel has multiple holes/perforations.

The second channel has multiple perforations, in the range of a few hundred, along its length, preferably commencing 5 to 75 cm from the distal end of the first channel. The third channel has multiple perforations, in the range of a few hundred; these perforations may be of the same size or variable size. If they are of variable size, their size may increase or decrease from the proximal to the distal end of the second channel. The third channel may not have any perforations while the second channel is continuing. Once the second channel ends, the third channel may have multiple perforations commencing at 0 cm or for example after 10-20 cm from the distal end of the second channel.

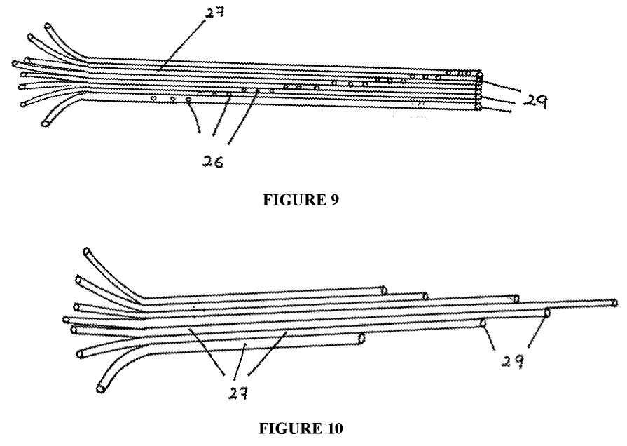

According to another embodiment of the present invention, the enteral feeding device is in the form of a more than three channel version wherein the second channel extends for some distance from the distal end of the first channel. The second channel has multiple perforations, in the range of a few hundred, along its length, preferably commencing from 0 to 75 cm from the distal end of the first channel. The third channel has multiple perforations, in the range of a few hundred; these perforations may be of the same size or variable size. If they are of variable size, their size may increase or decrease from the proximal to the distal end of the second channel. The third channel does not have any perforations while the second channel is continuing. Once the second channel ends, the third channel may have multiple perforations commencing at 0 cm or preferably after 10-20 cm from the distal end of the second channel. The same pattern is repeated for the subsequent channels. The third channel does not have any perforations while the second channel is continuing.
According to another embodiment of the invention as illustrated in FIG. 9, Long-length enteral feeding tube with multiple channels (27) (up to 100 channels) for insertion through nasogastric route, gastrostomy, jejunostomy or rectal route is provided. Each channel be left open at distal end or closed at the end with a side hole (26) near the end. The channels run parallel to each other for the entire length of the tube. Each channel (29) has multiple perforations for the exit of nutrients/medications. Each channel is open at the proximal end and is connected to delivery system for delivery of nutrients or medications.
Further, in another variant as illustrated in FIG. 10, channels run parallel to each other for variable length and there are no holes. Each channel can be left open at distal end or closed at the distal end with a side hole near the end. The channels run parallel to each other for variable length of the tube. The individual channels (29) have no side holes along their length except the hole near their end. This tube may be inserted through nasogastric route, gastrostomy, jejunostomy or rectal route. Each channel is open at the proximal end and is connected to delivery system for delivery of nutrients or medications.

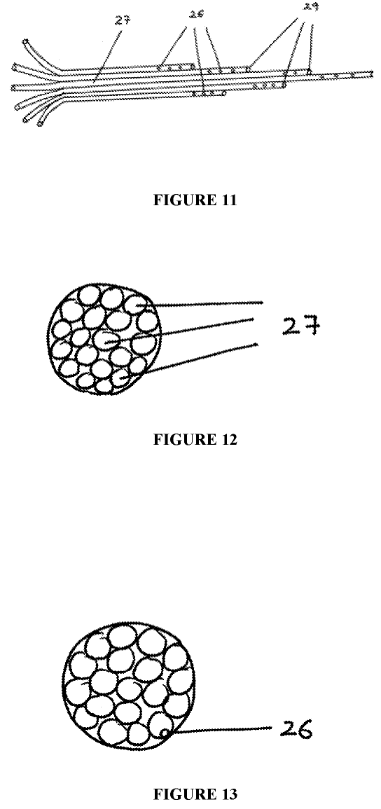

In another embodiment illustrated in FIG. 11, each channel can be left open at distal end or closed at the distal end with a side hole (26) near the end. The channels run parallel to each other for variable length of the tube. The individual channels (29) have multiple side holes (26) along their length. This tube may be inserted through nasogastric route, gastrostomy, jejunostomy or rectal route. Each channel is open at the proximal end and is connected to delivery system for delivery of nutrients or medications.

According to one embodiment of the present invention, the enteral feeding device comprises two channels, each with one opening at the distal end.

According to one embodiment of the present invention, the enteral feeding device comprises two channels with the first channel having a single opening at its distal end and with the second channel having multiple perforations commencing after a few centimetres, as illustrated in FIG. 1, from the distal end of the first channel.

The enteral feeding device may be inserted into the body through the naso or rectal route for those who require it for short or medium-term basis. Further, it can be inserted by a surgical opening in the stomach (Gastrostomy) or Jejunum (Jejunostomy) after certain modifications, for those who require it on a long-term basis.

According to one embodiment of the present invention, the enteral feeding device may be inserted in the patient through the naso-gastric route as illustrated in FIGS. 14 and 16. The device in this case consists of the following:
a guide wire through the gastric lumen (1),
a guide wire through the jejuno-ileal lumen (4),
a lumen each for stomach (33) and jejunum (35) and ileum (36) of small intestine,
common channel (9) enclosing both the tubes,
an opening in the first lumen for the stomach (33), and
second lumen for small bowel (15,19) with the first few centimetres, as mentioned hereinabove, from distal end of first lumen without perforations and remaining portion of lumen having multiple perforations.

According to another embodiment of the present invention, the enteral feeding device may be inserted in the patient through gastrostomy as illustrated in FIGS. 6 and 15 or jejunostomy. The device in this case consists of the following:
a guide wire through the gastric lumen (1),
a guide wire through the jejuno-ileal lumen (4),
a lumen each for stomach (33) and jejunum (35) and ileum (36) of small intestine,
common channel (9) enclosing both the tubes,
an opening in the first lumen for the stomach (33),
second lumen for small bowel (15,19) with the first few centimetres, as mentioned hereinabove, from distal end of first lumen without perforations and remaining portion of lumen having multiple perforations,
a ring (20) which is capable of sliding freely on the enteral feeding channel, is used to keep the channel in position outside the body on the abdominal wall,
a hollow tube referred to as T bar (21), which can slide freely on the enteral feeding channel, has a hole which is perpendicular to the lumen of the T bar (21), through which the enteral feeding channel passes, and
a disc shaped structure (22) fixed to the channel, which is positioned inside the wall of the stomach (33). Said structure along with the sliding ring (20) and T bar (21) helps fix the channel in gastrostomy or jejunostomy insertions.

In another embodiment, the enteral feeding device is capable of withdrawing fluid including nutrients and/or medications from multiple, discrete points in the GI tract through multiple channels (27) and multiple perforations. This withdrawn fluid including nutrients and/or medications may be useful in investigating and evaluating the digestion and absorption in the Gastro-intestinal tract.

In yet another embodiment, the enteral feeding device is capable of re-inserting the withdrawn nutrients and/or medications through different channels and perforations to aid digestion and/or absorption in various other parts of the GI tract.

In a further embodiment, the enteral feeding device has conducting wires (40) running along the entire length or part of its length.

In one embodiment, the conducting wires (40) are on the outer surface of the enteral feeding device as illustrated in FIGS. 18 and 19. In FIG. 19, the conducting wire (40) has connectors (41) which send signals to the gates (42,43) that control the openings in the device/channels. These gates (42) are open and the signals sent through the conducting wire (40), and their wire connectors (41) can close the gates (43). The gates, when closed, prevent any fluid leaking from the lumen of the tube (24).

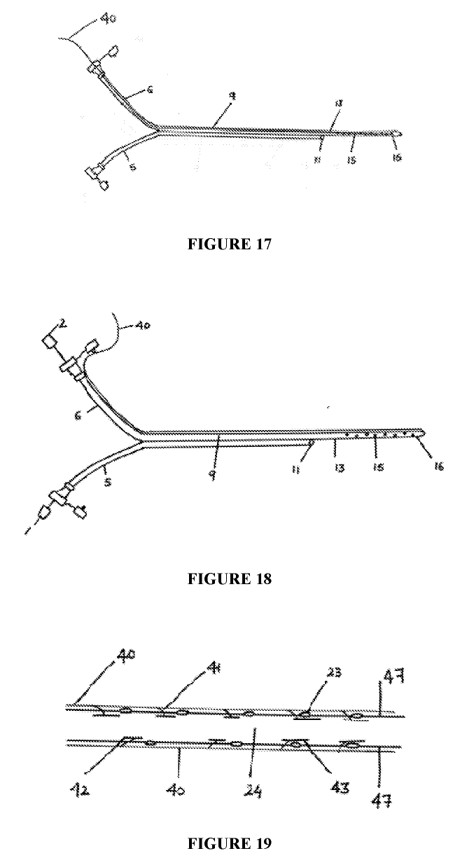

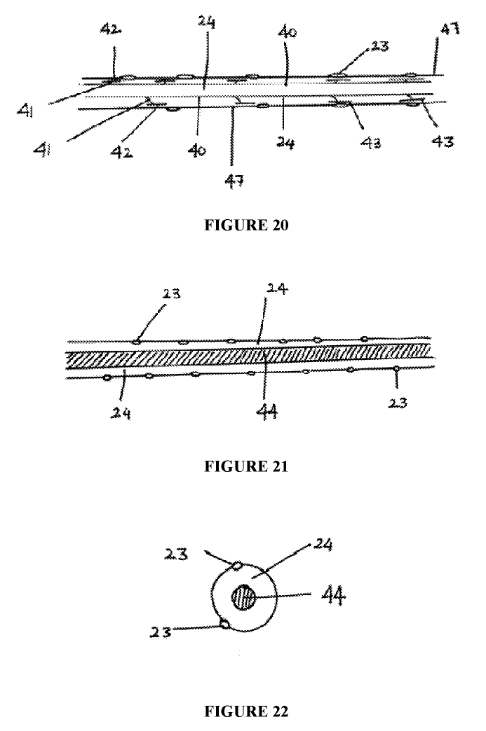

In another embodiment, the conducting wires (40) are inside the enteral feeding device as illustrated in FIGS. 17 and 20. In FIG. 20, the conducting wire (40) has connectors (41) which send signals to the gates (42,43) that control the openings in the device/channels. These gates are open (42) and the signals sent through the conducting wire (40) and the connectors (41) can close the gates (43). The gates, when closed, prevent any fluid leaking from the lumen of the tube (24).

In yet another embodiment of the enteral feeding device, conducting wires (40) have one or more sensors for measuring pressure, temperature, pH and other like parameters.

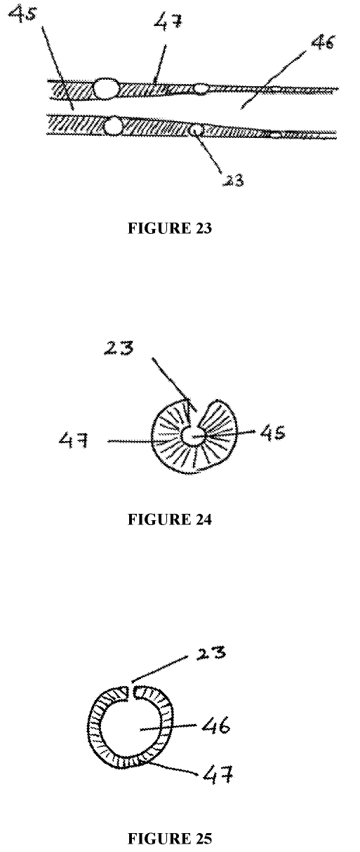

The conducting wires (40) serve the following purposes:
a) Ability to send electrical signals: These signals are responsible for the following functions:
i) to facilitate the closing and opening of various perforations/openings at various times by sending electrical signals to the gates. The openings in the channel have ‘gates’ (42,43) regulating their opening and closure as illustrated in FIGS. 19 and 20. Thus, these gates (42,43) have an important regulatory function. In a single channel tube with multiple perforations or a tube with multiple channels (27) with multiple perforations, gates (42,43) allow some perforations in a channel to open while other perforations are closed and at other times, gates (42,43) allow the opening of new perforations, while closing the previous perforations. This enables more effective distribution of the nutrients and/or medication and their withdrawal from the lumen of the intestine. The electrical signals that co-ordinate opening and closing of the various gates (42,43) are sent through the conducting wires (40) to the gates (42,43).
ii) The electrical signals delivered by conducting wires (40) can stimulate movement in any localised part of the intestine, thereby facilitating effective absorption of the nutrients and/or medication through the surrounding parts of the intestine.
(b) Sensors for detection: These conducting wires (40) have sensors which help to measure pressure, temperature, pH and other like parameters.
As explained above, the wires can also have fibro-optic sensors which can help in transmitting light to and fro in the tube. The light reflected back from the sensors in the tube or lumens will be analysed by a device attached to the enteral tube outside the body. This device will give measurements of pH (Acidity vs Alkalinity), temperature and pressure and curvature. Some of these measurements such as pH of the tube can help in determining the position of the tip of the tube as explained in the FIGS. 26-31. They can also help in detecting blockage of the tube (be it main channel or any of the multiple channels at an early stage leading to remedial measures. These fibre-optics wires are significant and can be inserted in all kinds of feeding tube versions such as but not limited to nasogastric, nasojejunal, gastroduodenal, gastrojejunal and jejunal versions.
In another embodiment of the present invention, the lumen of the tube (24) and various channels is of variable internal diameters to facilitate the uniform distribution of fluids including nutrients and/or medication as illustrated in FIGS. 23, 24 and 25. The lumen of the tube (24) or its channels have wall (47) thickness of variable dimensions, giving a narrow lumen (45) at one place and a wide lumen (46) at another place. There are holes (23) in the wall of the tube (47).
In a further embodiment, the lumen of the tube (24) and various channels has a solid core (44) inside for facilitating uniform distribution of fluids including nutrients and/or medication exiting through the perforations as illustrated in FIGS. 21 and 22. In FIGS. 21 and 22, the solid core (44) allows fluids to flow in concentric fashion in the lumen of the tube (24) around the solid core (44) to allow uniform distribution of the fluids from the holes (23) in the lumen of the tube (24) into the lumen of the intestine.

In yet another embodiment of the present invention, the lumen of the tube (24) and various channels has a solid core (44) inside which runs along the entire length of the device.

In yet another embodiment of the present invention, the lumen of the tube (24) and various channels has a solid core (44) inside which runs along the distal part of the device.

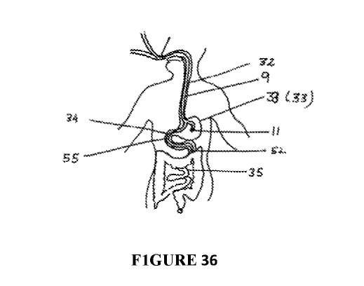

In still further embodiment an enteral tube with double lumen for nasogastric route is provided (FIG. 36). This tube passes through the oesophagus (32) in which (both the lumens run parallel to each other (9). One lumen opens into the stomach (33) with a single opening (11). The second channel of the tube goes beyond the outlet of the stomach into the duodenum (34), it can go further into jejunum (35) too. The part of the tube that runs through the duodenum/jejunum (55) can have no side hole or multiple side holes, however this tube will have a hole near its end (52).

The utility and the application of the present invention are illustrated by means of the following:
(1) Gastroschisis is a birth defect of the abdominal wall. The infant's intestines protrude out of the body, through a hole beside the umbilicus. Sometimes other organs, such as the stomach and liver may also protrude out of the infant's body. One out of 2500 to 5000 children are born with this defect. The Centres for Disease Control and Prevention (CDC) estimates that about 1,871 babies are born each year in the United States with gastroschisis. A baby born with this defect is required to undergo surgery soon after birth, in order to place the abdominal organs inside the baby's body and repair the abdominal wall. Even after the repair, infants with gastroschisis can have problems with feeding, digestion of food and absorption of nutrients. For a period of almost 8 weeks subsequent to the surgery, the gastrointestinal tract is in ileus (a disruption of the normal propulsive ability of the gastrointestinal tract) and the child cannot be fed by the oral route or nasogastric route (via nasogastric tube).

Subsequent to the operation, a nasogastric tube is always left in the stomach to facilitate drainage of its secretions. Therefore, in this situation, where the entire GI tract of an infant is affected by ileus post-operatively, the present invention is able to simultaneously supply a small amount of nutrients (termed as ‘trophic feed’) and/or medications to multiple discrete parts of the GI tract despite the absence of peristalsis, enhancing recovery of the infant, preserving liver function and luminal nutrition, while also enabling aspiration of gastric secretions. The stomach is the last part of the GI tract to recover from ileus. In the later part of the recovery phase from paralytic ileus, the present invention facilitates the delivery of a larger amount of nutrients (non-trophic feeds) into the functioning small intestine (with normal motility/peristalsis) while still enabling aspiration of gastric secretions.

It is relatively easy to insert the naso-enteral tube as disclosed in the present invention and start nutrition on the second day post-operatively. This could result in saving the lives of more than one hundred thousand babies in developing countries, where people cannot afford safe TPN due to high cost and sophisticated preparation involved in its delivery and monitoring. Currently most of the babies born with this defect die in the developing world due to lack of nutrition. Therefore, this group of patients will be benefited directly with the present invention.

In developed countries, TPN costs approximately £250-£700 per day. Furthermore, the use of TPN leads to liver dysfunction as enteral nutrition cannot be administered to the infant patient till 2 to 4 weeks post-surgery. Use of the present invention in developed countries may help to preserve liver function and facilitate earlier delivery of nutrition to the intestinal lumen. This could enhance recovery of intestinal motility and function, thereby decreasing the period that these children require TPN.
(2) In cases where infants are born with a pseudo-obstruction with defective gut motility, the GI tract lacks peristalsis. An intestinal transplant is usually done to save the life of the child. Post-surgery, the stomach and the small and large intestines lack motility. These patients are required to be given life-long TPN. The present invention, in such circumstances delivers nutrients and/or medications to multiple discrete points in the GI tract. Simultaneous supply of nutrients to various parts of the GI tract helps the infant to recover at a faster rate. The long-length enteral tube as disclosed in the present invention can particularly benefit this class of patients. They will save the high cost of life-long TPN and their life expectancy will also increase, as with the administration of nutrients through the enteral tube as disclosed in the present invention their liver and inner lining of the intestines will be preserved. Therefore, this invention could help save many lives.
(3) The GI tract of many patients afflicted with a spinal cord injury suffers from lack of peristalsis. In this condition also the present invention aids in speedy recovery by supplying nutrients and medications to multiple and discrete points in the GI tract.
   The examples and embodiments disclosed in the specification are illustrative in nature and should not be deemed to limit the scope or extent of the present invention in any manner whatsoever. Any modifications/variants of the disclosed embodiments and examples which are apparent to a person ordinarily skilled in the art should be read as part and parcel of the present invention.

1. An enteral feeding tube capable of delivering nutrients and/or medications to a patient at multiple points in the gastrointestinal tract and/or aspirating the gastrointestinal tract, comprising two or more channels each with at least one opening, said channels running parallel to the other until one channel ends, each channel keeping the fluid separated from the other channel, said channels being open at the proximal end and closed or open at the distal end and each channel having one or more perforations of same or variable size.

2. An enteral feeding tube capable of delivering nutrients and/or medications to a patient at multiple points in the gastrointestinal tract and/or aspirating the gastrointestinal tract, comprising two or more channels each with at least one opening, said channels running parallel to the other until one channel ends, each channel keeping the fluid separated from the other channel, said channels being open at the proximal end and closed or open at the distal end and each channel having one or more perforations of same or variable size, one or more channels having fibro-optic sensors for temperature, pH and pressure determination.

3. The enteral feeding tube as claimed in claim 2, comprising fibro-optic sensors for light-based timely detection and prevention of migration/displacement of the tube to an unintended location.

4. The enteral feeding tube as claimed in claim 2, comprising fibro-optic sensors for light-based timely detection and prevention of blockage of said tube.

5. An enteral feeding tube being a two lumen tube with one lumen extending till the stomach and the other lumen extending beyond the stomach.

6. The enteral feeding tube as claimed in claim 1, wherein lumen is formed by separate channels being enclosed in a common tube.

7. The enteral feeding tube as claimed in claim 1, wherein lumens are formed by creating partitions longitudinally in the tube.

8. The enteral feeding tube as claimed in claim 1, wherein the tube is made of any flexible and biocompatible material, such as but not limited to silicone, polyvinylchloride, polyurethane.

9. The enteral feeding tube as claimed in claim 1, wherein said tube ranges from 25 to 1000 cm in length.

10. The enteral feeding tube as claimed in claim 1, wherein length of the first channel is in the range of 5 to 50 cm and is provided with one or more perforations.

11. The enteral feeding tube as claimed in claim 1, wherein length of the first channel is alternatively in the range of 10 to 50 cm and is provided with one or more perforations.

12. The enteral feeding tube as claimed in claim 1, wherein the tube consists of two channels with length of second channel in the range of 25 to 1000 cm from the distal end of the first channel.

13. The enteral feeding tube as claimed in claim 1, wherein the tube consists of three channels with length of second channel in the range of 25 to 1000 cm from the distal end of the first channel.

14. The enteral feeding tube as claimed in claim 1, wherein length of the second channel is in the range of 10 to 1000 cm from the distal end of the first channel.

15. The enteral feeding tube as claimed in claim 1, wherein the second channel has multiple perforations preferably commencing at a distance of 0 to 100 cm from the distal end of the first channel.

16. The enteral feeding tube as claimed in claim 1, wherein the second channel has multiple perforations commencing at a distance of 5 to 50 cm from the distal end of the first channel.

17. The enteral feeding device as claimed in claim 1, wherein channels subsequent to second channel have multiple perforations preferably commencing at a distance of 0 to 100 cm from the distal end of the second channel.

18. The enteral feeding tube as claimed in claim 1, wherein the third channel has multiple perforations commencing from the distal end of the second channel.

19. The enteral feeding tube as claimed in claim 1, wherein the third channel has multiple perforations at a distance of 0 to 100 cm from the distal end of the second channel.

20. An enteral feeding tube capable of delivering nutrients and/or medications to a patient at multiple points in the gastrointestinal tract and/or aspirating the gastrointestinal tract, comprising two or more channels each with at least one opening, said channels running parallel to each other for the whole length of the tube, i.e. they end at the same length, each channel keeping the fluid separated from the other channel, said channels being open at the proximal end and closed or open at the distal end and each channel having one or more perforations of same or variable size.

21. The enteral feeding tube as claimed in claim 20, wherein the tube has up to 100 channels and each channel has multiple perforations along its length for the exit/entry of nutrients and/or medications into the gastrointestinal tract and said perforations are of variable number, ranging from one to hundred.

22. An enteral feeding tube capable of delivering nutrients and/or medications to a patient at multiple points in the gastrointestinal tract and/or aspirating the gastrointestinal tract, comprising two or more channels each with at least one opening, said channels running parallel to each other but ending at different points along the length of the tube, wherein each channel is open at both ends, each channel keeping the fluid separated from the other channel.

23. The enteral feeding tube as claimed in claim 22, wherein the tube has up to 100 channels and each channel further comprises one or more perforations along its length, wherein the perforations are of same or variable size and of variable number, ranging from one to hundred.

24. The enteral feeding tube as claimed in claim 22 wherein the channels are closed at one end.

25. The enteral feeding tube as claimed in any claim 1, wherein said tube is capable of being used for short or medium duration by insertion through the nasal or rectal cavity.

26. The enteral feeding tube as claimed in claim 1, wherein said tube is capable of being used for long duration by gastrostomy or jejunostomy, after modification.

27. An enteral feeding tube capable of insertion in the patient through the naso-gastric route, comprising:
two or more tubes each with at least one lumen, said tubes running parallel to the other until one tube ends, each tube keeping the fluid separated from the other tube, said tubes being open at the proximal end and closed or open at the distal end and each tube other than the first tube having one or more holes of same or variable size,
a lumen each for stomach (33) and jejunum (35) and ileum (36) of small intestine,
common tube enclosing the channels,
the first opening in the first channel for the stomach (33), and
the second set of openings for the small intestine, including multiple holes along the length of the channel, which start 1-50 cm from the end of the channel ending in the stomach (33).

28. An enteral feeding tube capable of insertion by gastrostomy or jejunostomy, comprising:
two or more tubes each with at least one lumen, said tubes running parallel to the other until one tube ends, each tube keeping the fluid separated from the other tube, said tubes being open at the proximal end and closed or open at the distal end and each tube other than the first tube having one or more perforations of same or variable size,
a lumen each for stomach (33) and jejunum (35) and ileum (36) of small intestine,
common channel (9,10) enclosing the tubes,
an opening in the first lumen for the stomach (33),
second lumen for small bowel (15, 19) not perforated at the proximal end,
a guide wire through the gastric lumen (1),
a guide wire through the jejuno-ileal lumen (4),
a ring (20),
a hollow tube (21), and
a disc shaped structure (22).

29. The enteral feeding tube as claimed in claim 28, wherein the ring (20) is capable of sliding freely on the enteral feeding tube to keep the tube in position outside the body on the abdominal wall.

30. The enteral feeding tube as claimed in claim 28, wherein the hollow tube (21) comprises of a hole which is perpendicular to the lumen of the hollow tube (21), through which the enteral feeding tube passes.

31. The enteral feeding tube as claimed in claim 28, wherein the hollow tube (21) is capable of sliding freely on the enteral feeding tube.

32. The enteral feeding tube as claimed in claim 28, wherein the disc shaped structure (22), fixed to the tube, is positioned inside the gastrointestinal tract.

33. The enteral feeding tube as claimed in claim 28, wherein the disc shaped structure (22) along with the sliding ring (20) and hollow tube (21) enable fixing of the tube in gastrostomy or jejunostomy insertions.

34. The enteral feeding tube as claimed in claim 1, wherein said tube is capable of being shortened by cutting at proximal or/and distal end to tailor its length to fit patients of various sizes and builds.

35. The enteral feeding tube as claimed in claim 1, wherein the two or more channels with one or more perforations can be used to withdraw fluid including nutrients and/or medication from various parts of the intestine.

36. The enteral feeding tube as claimed in claim 1, wherein the withdrawn fluid can be evaluated for digestion or re-inserted through other perforations in other channels to aid digestion and absorption of nutrients and/or medications in different parts of the GI tract.

37. The enteral feeding tube as claimed in claim 1, having conducting wires (40) running along the entire length or part of its length.

38. The enteral feeding tube as claimed in claim 37, wherein the conducting wires (40) are on the outer surface of the enteral feeding tube.

39. The enteral feeding tube as claimed in claim 37, wherein the conducting wires (40) are inside the enteral feeding tube.

40. The enteral feeding tube as claimed in claim 37, wherein the conducting wires (40) have the ability to send electrical signals to gates (42,43) regulating the opening and closing of the perforations and controlling the distribution of nutrients and/or medication in the lumen of the GI tract.

41. The enteral feeding tube as claimed in claim 37, wherein the conducting wires (40) have the ability to send electrical signals to stimulate movement in a localised part of the intestine to facilitate digestion and absorption of nutrients and/or medication.

42. The enteral feeding tube as claimed in claim 37, wherein the conducting wires (40) have one or more sensors to measure temperature, pressure, pH and other like parameters.

43. The enteral feeding tube as claimed in claim 37, wherein the conducting wires (40) have one or more fibro-optic sensors to measure temperature, pressure and pH.

44. The enteral feeding tube as claimed in claim 37, wherein the conducting wires (40) have one or more fibro-optic sensors for light-based timely detection of migration/displacement of the tube to an unintended location.

45. The enteral feeding tube as claimed in claim 37, wherein the conducting wires (40) have one or more fibro-optic sensors for light-based timely detection blockage of the tube.

46. (canceled)

47. The enteral feeding tube as claimed in claim 1, wherein the lumen of the tube (24) is of variable internal diameters to facilitate the uniform distribution of fluids including nutrients and/or medication in the lumen of the GI tract.

48. The enteral feeding tube as claimed in claim 1, wherein the lumen of the tube (24) has a solid core (44) inside for facilitating uniform distribution of fluids including nutrients and/or medication exiting through the perforations.

49. The enteral feeding tube as claimed in claim 47, wherein the lumen of the tube (24) has a solid core (44) inside which runs along the entire length of the tube.

50. The enteral feeding tube as claimed in claim 47, wherein the lumen of the tube (24) has a solid core (44) inside which runs along the distal part of the tube.

51. An enteral feeding tube capable of insertion in the patient through a naso-gastric route wherein the tube has two lumens, one lumen that helps in drawing out fluid from a stomach and one lumen which can help in introduction of feeds in part of an intestine beyond the stomach.

52. The enteral feeding tube as claimed in claim 51 wherein the tube has one or more openings in the lumen.

53. The enteral feeding tube capable of insertion in the patient through the naso-gastric route as claimed in claim 27, wherein the tube has multiple lumens.

54. The enteral feeding tube as claimed in claim 37, wherein the conducting wire is a fibro-optic wire.

55. (canceled)

56. The enteral feeding tube as claimed in claim 27, wherein the tube has one or more openings towards the distal end of the channel.

57. The enteral feeding tube as claimed in claim 27, wherein the tube is inserted into the gastrointestinal tract using a guide wire through the gastric lumen (1) or through using a guide wire through the jejuno-ileal lumen (4).

58. The enteral feeding tube as claimed in claim 48 wherein the solid core is in the form of guidewire and can be inserted and pulled out of the lumen and/or channels of the tube.

59. An enteral feeding tube capable of insertion in the patient through the naso-gastric route comprising two lumens wherein two lumens run parallel to each other (9) wherein one lumen opens into the stomach (33) with a single opening (11) and the second lumen goes beyond the outlet of the stomach into the duodenum (34) or jejunum (35) and has no side hole or multiple side holes, but has a hole near its end (52).
